(19) United States
(12) Patent Application Publication
(10) Pub. No.: US 2004/0054378 A1
(43) Pub. Date: Mar. 18, 2004

(54) Set of surgical instruments for carpal tunnel surgery

(76) Inventor: Cher-Chung Yang, Tainan (TW)

Correspondence Address: Dennison, Schultz & Dougherty, 612 Crystal Square 4, 1745 Jefferson Davis Highway, Arlington, VA, 22202-3417, US

(21) Appl. No.: 10/244,553

(22) Filed: Sep. 17, 2002

(51) Int. Cl. A61M 29/00

(52) U.S. Cl. 606/191

(57) ABSTRACT

A set of surgical instruments for carpal tunnel surgery includes a group of differently-sized dilators for progressively dilating a carpal tunnel of a patient. A pair of guides is adapted to be inserted in the dilated carpal tunnel. Each of the guides has a trough defining a first longitudinal and a second longitudinal groove, and the trough is formed with a rear handle defining a cavity open to the first longitudinal groove. A group of surgical knives is each movable along the second longitudinal groove of the guide for cutting. Each of the surgical knives is provided with a shank in a zigzag configuration, and the shank has a rear end formed with a grip and a front end formed with an angled head.

### BACKGROUND OF THE INVENTION

1. Field of the Invention

The present invention relates to a set of surgical instruments and, more particularly, to a set of surgical instruments for carpal tunnel surgery.

2. Description of Related Art

Many professions necessitate frequently-repeated motions of the hands, such as secretaries, computer operators, carpenters, dentists and the like. Such hand motions especially repeated flexions in the wrist and the fingers usually bring the palm into a pathological condition known as carpal tunnel syndrome, which indicates a disease resulted from a constricted median nerve in the palm. A numb sensation at the palm and fingers is the cardinal symptom of the disease.

The occupational disease has proved difficult to be prevented and surgery is required if there is no improvement as a result of six weeks' conservative treatment.

As shown in FIG. 6, the surgery in early times was carried out firstly by making an incision about 4˜5 centimeters in the palm skin, so as to cut the palmar longus fascia and the transverse ligment (70) until both a motor branch (73) and a median nerve (71) constricted by the swollen transverse ligment (70) are exposed to view. It was from then on that abnormally conglutinated tissues in the carpal tunnel, especially those around the median nerve (71), were separated. The surgery would take 40 to 50 minutes because much care would be taken in order not to cut other nerves by accident. It would also take a prolonged time for the cut in the palmar longus fascia as well as the long incision in the palm to heal up.

In recent years, endoscopes are involved in such surgery to solve the above-mentioned problems. The surgery is now carried out by making only a short incision in the wrist instead of in the palm. Because the incision is made deep enough to extend to the carpal tunnel, dilators can be squeezed into the tunnel to separate abnormally conglutinated tissues and thus progressively dilate the carpal tunnel to such a size that a guide (50) as shown in FIG. 7 may be inserted therein.

The guide (50) has a longitudinal groove (51) for guiding both an endoscope (not shown) and a surgical knife (60). The knife (60) is then moved along the guide (50) from the wrist to the palm to cut the swollen transverse ligament under surveillance by the endoscope. This surgery takes only a short time and the short incision will heal up soon.

On the other hand, the guide (50) is a disposable implement usually made of plastic to avoid infection among patients, and the single groove (51) requires the blade of the surgical knife (60) to be moved ahead of the endoscope during the operation. Because the knife (60) is made in a straight configuration, its blade is inevitably moved in a direction at a certain angle with the groove (51) while cutting. So the blade cuts the soft wall of the groove (51) and leaves the resulting chips in the carpal tunnel. These chips are a potential danger which will result in inflammation a short time later due to foreign body reaction.

Therefore, it is an objective of the invention to provide a set of surgical instruments to mitigate and/or obviate the aforementioned problems.

### SUMMARY OF THE INVENTION

The object of the present invention is to provide a set of surgical instruments in which no chip will be made and left in the carpal tunnel of a patient.

Another object of the present invention is to provide a set of surgical instruments in which a surgical knife can be moved smoothly during the operation.

Still another object of the present invention is to provide a set of surgical instruments in which excellent surveillance can be made upon the entire operation.

Other objects, advantages and novel features of the invention will become more apparent from the following detailed description when taken in conjunction with the accompanying drawings.

### BRIEF DESCRIPTION OF THE DRAWINGS

FIG. 1 is a view of a set of surgical instruments in accordance with the present invention for carpal tunnel surgery;

FIG. 2 is a sectional view showing a surgical knife and a guide both included in the set of surgical instruments of FIG. 1;

FIG. 3 is a schematic view showing a variable distance from a blade of the surgical knife to the guide;

FIG. 4 is a transverse sectional view of the guide of FIG. 2;

FIG. 5 is a perspective view showing the surgical knife and the guide of FIG. 2 during operation;

FIG. 6 is a view of a human hand, showing tissues involved therein; and

FIG. 7 is a perspective view illustrating a set of conventional surgical instruments including a straight surgical knife and a guide.

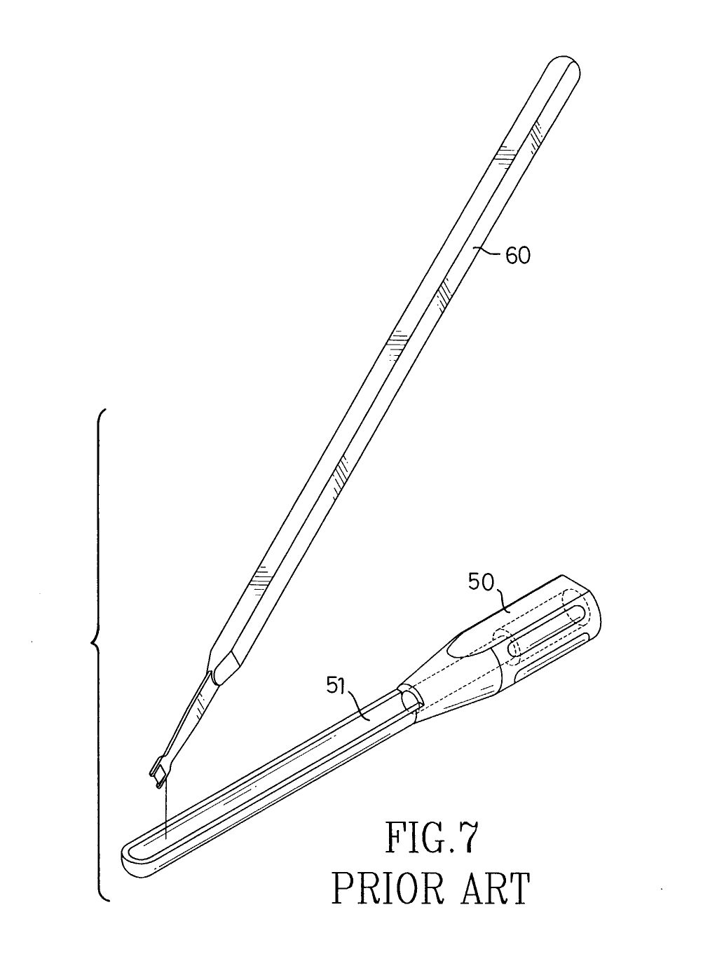

### DETAILED DESCRIPTION OF THE PREFERRED EMBODIMENT

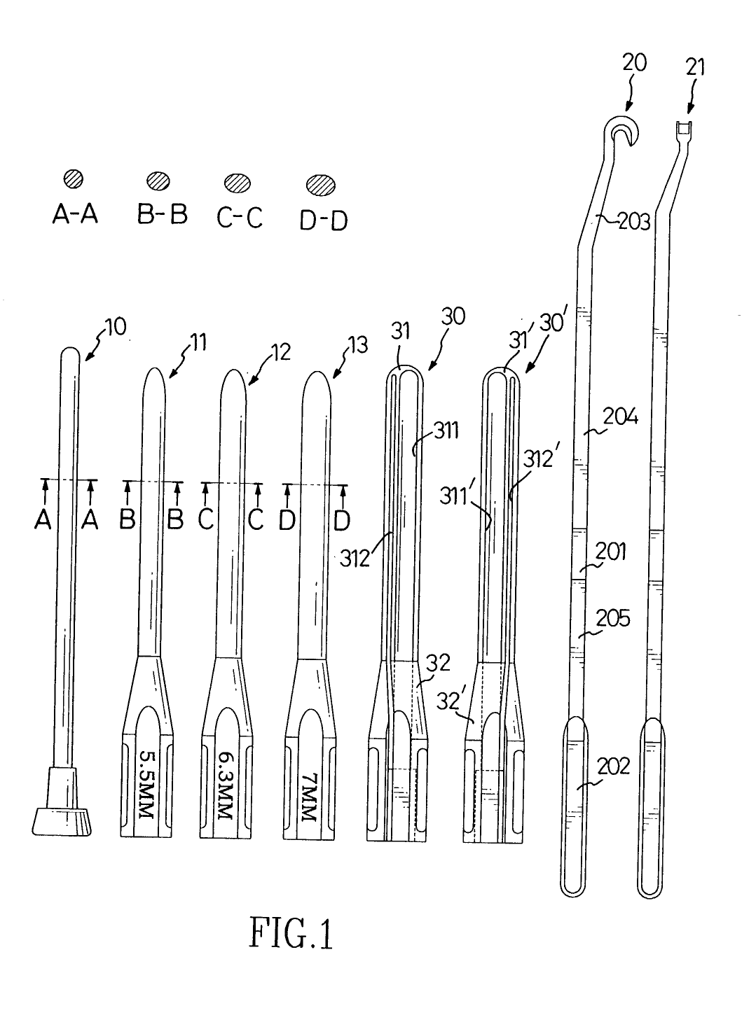

Referring to FIG. 1, there is shown a set of surgical instruments in accordance with the present invention for carpal tunnel surgery. The inventive set of instruments includes a group of four differently-sized dilators (10, 11, 12, 13): one (10) has a round section as shown in a sectional view designated by A-A, and the others (11, 12, 13) each have an oval section as shown in sectional views designated by B-B, C-C and D-D. These dilators (10, 11, 12, 13, 14) are provided for progressively dilating the patient's carpal tunnel, which is oval in shape.

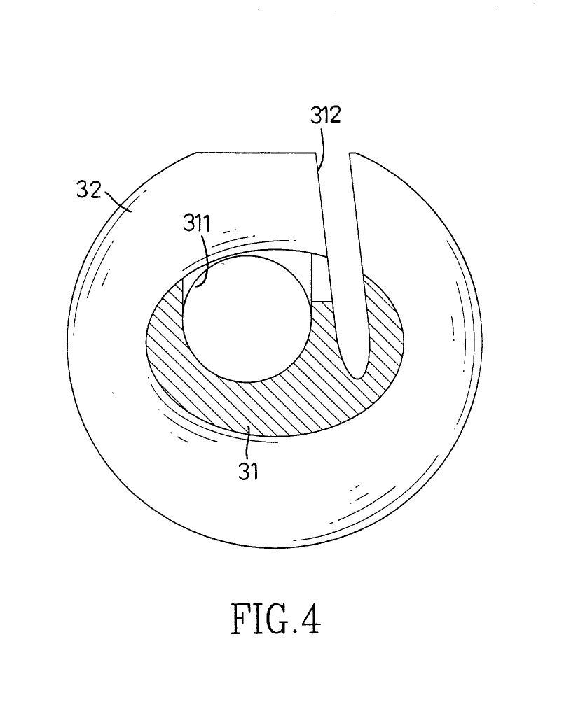

A pair of guides (30, 30′), one for the left hand of a patient and the other for the right hand, are each designed to be inserted in the properly dilated carpal tunnel. Each of the guides (30, 30′) has a trough (31, 31′) formed with a rear handle (32, 32′), with a first longitudinal groove (311, 311′) and a second longitudinal groove (312, 312′) defined in the trough (31, 31′). Preferably, the second groove (312, 312′) extends through the full length of the rear handle (32, 32′). The handle (32, 32′) of the guide (30, 30′) is formed with a cavity (not numbered) open to the first longitudinal groove (311, 311′), as best seen in FIG. 4.

There is further provided a group of surgical knives (20, 21), including a first knife (20) having a hooked blade, which preferably has a blunt outer edge as a knife back, and a second knife (21) having a short straight blade.

Since the guides (30, 30′) are symmetrical with respect to one another and the two knives (20, 21) are similar to each other except for their blades, description will be made only on one of the guide (30) and the first knife (20) hereinafter.

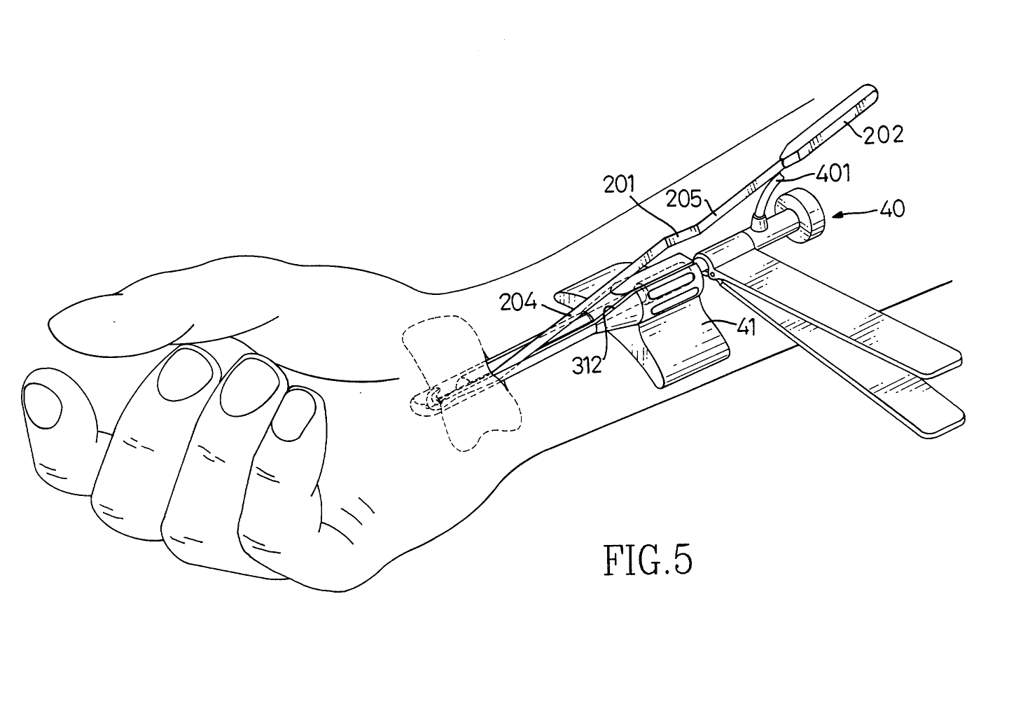

The first surgical knife (20) includes a shank (201) having a rear end formed with a grip (202) and a front end formed with an angled cutting head (203). In fact, the shank (201) consists of a flectional front section (204) and a straight rear section (205) that are interconnected to provide the shank (201) with a zigzag configuration, as shown in FIG. 5.

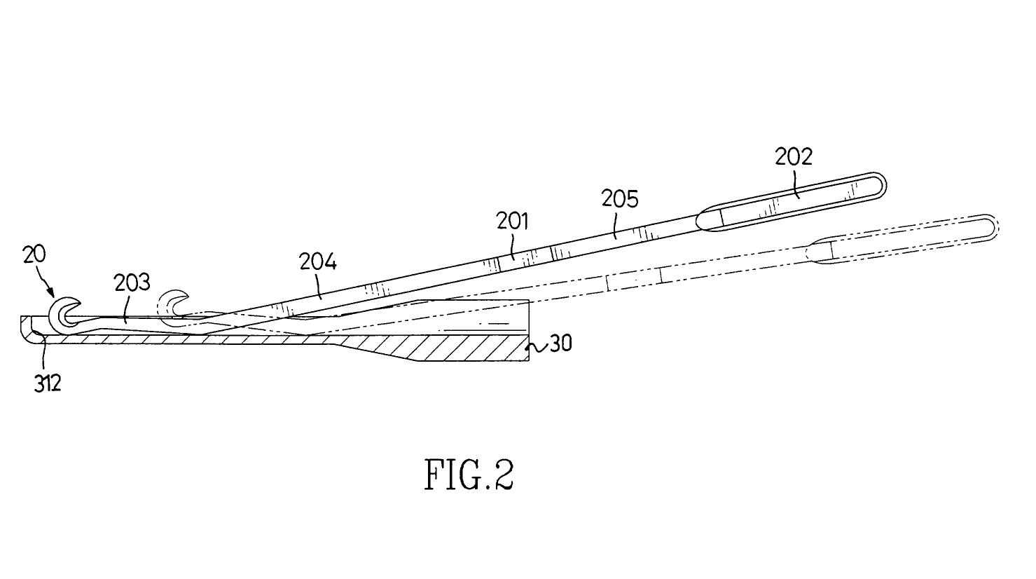

From a geometric standpoint, the angled cutting head (203) and the straight rear section (205) are in the same reference plane as FIG. 1 is in, but the flectional front section (204) is in a plane just perpendicular to the reference plane. The perpendicular relationship between the planes makes the zigzag shank (201) appear straight in FIG. 1 as well as in FIG. 2, though it is not really so.

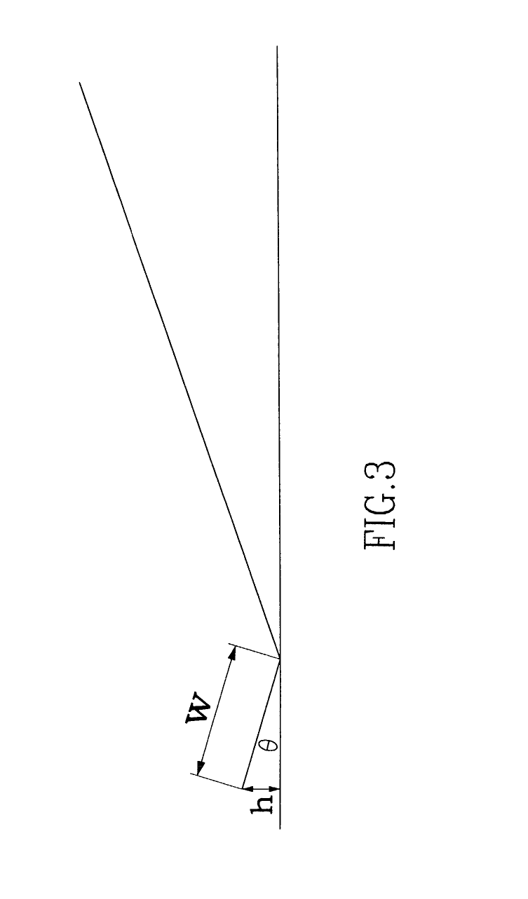

Referring to FIGS. 2 and 3, the angled head (203) of the surgical knife (20) is movable along the guide (30) pulling inward from the distal site of the transverse ligament (70) in the second longitudinal groove (312) for the purpose of cutting the swollen transverse ligament (70) in the palm. The knife (20) may also be turned around a projecting juncture between the shank (201) and the angled cutting head (203), so as to adjust the distance from the blade of the angled cutting head (203) to the guide (30).

The distance h from the blade to the guide (30) is determined by the formula as follows:

h=w×sin θ

wherein w represents the length of the angled head (203) of the surgical knife (20) and θ stands for the angle between the angled head (203) and the guide (30).

Because the angle θ is variable, the blade carried on the angled head (203) can reach any point in a large region where the swollen transverse ligament extends.

Referring to FIG. 5, the operation is now performed by placing the smallest dilator (10) in one of the guides (30, 30′), which, together with the dilator (10), is then inserted into the dilated carpal tunnel through an incision made in the wrist.

The alternative one of the guides (30, 30′) to be inserted is selected so that its second groove (30, 30′) is on the little-finger-side (ulnar-side) of the hand on which the surgery is to be carried out. In short, the guide (30) is selected for the right hand, as clearly shown in FIG. 4, and the guide (30′) is selected for the left hand. In the illustrated example it is the guide (30) that is selected only for the purpose to be mentioned below.

After the insertion of the guide (30), the dilator (10) is removed before an endoscope (40) is pushed into the first longitudinal groove (311) of the guide (30) through the cavity in the handle (32). Then, a clip is held around the endoscope (40) as a grip for easy control of the movement of the endoscope (40) along the first groove (311). Optionally, a pad (41) may be placed beneath the handle (32) to keep the guide (30) motionless on the patient's arm.

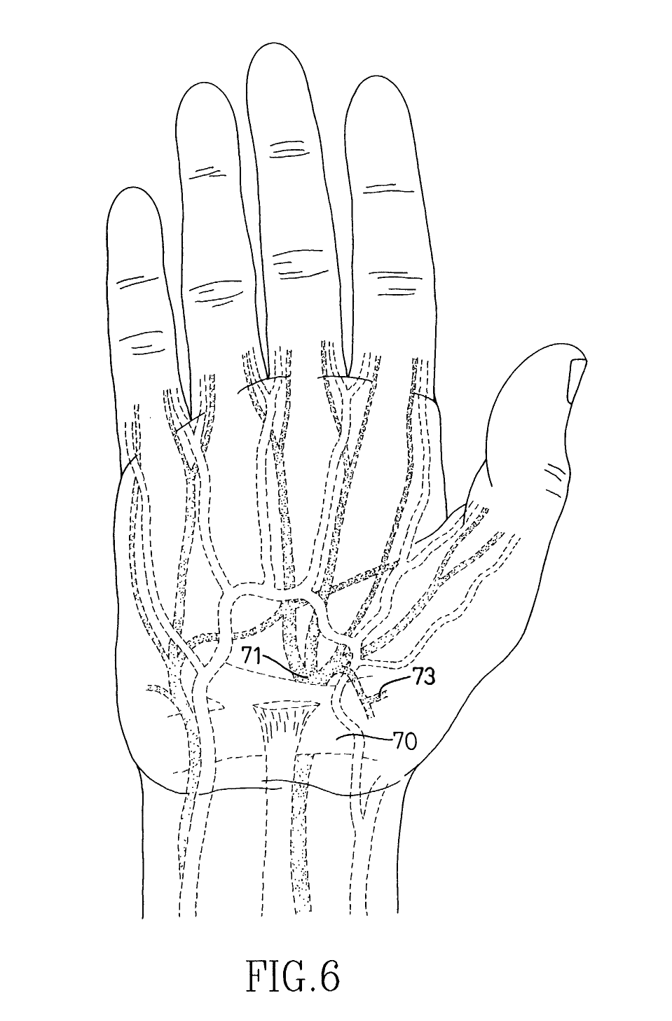

The angled cutting head (203) of the surgical knife (20) can be put into the second groove (312) at any point, including the part of the groove (312) defined in the handle (32), and is moved deep into the carpal tunnel for the operation, i.e. cutting the swollen transverse ligament in the palm under surveillance by the endoscope (40). In addition to the endoscope (40), the correctly selected guide (30) can avoid the injury of the knife (20) to the motor branch (73), as is shown in FIG. 6.

The angled cutting head (203) of the knife (20) is always alongside the endoscope (40) because the guide (30) provides two parallel, independent narrow grooves (311, 312). The two parallel grooves (311, 312) of the guide (30) facilitate the performance of the operation and the surveillance upon the same operation.

Also during the operation, the shank (201) designed in the zigzag configuration allows it to be kept away from an optical fiber line (401) of the endoscope (40), thereby ensuring the smooth movement of the surgical knife (20).

From the above description, it is noted that the invention has the following advantages:

1. High Safety in the Operation:

Because the guides (30) is provided with two independent grooves (311, 312) for the surgical knifes (20) and the endoscope (40), the angled head (203) of the knife (20) can be moved unobstructedly along the related groove (312) without cutting walls of the groove (312) and leaving resulting chips in the subcutaneous carpal tunnel.

2. Smooth Movement for the Surgical Knife (20).

Because the shank (201) is designed into the zigzag configuration, it is allowed to be kept away from the optical fiber line (401) of the endoscope (40) and so the surgical knife (20) can be moved smoothly.

3. Excellent Surveillance upon the Operation:

Because the endoscope (40) is always alongside the angled head (203) of the surgical knife (20), it can take a clear view of the blade on the head (203) and so an excellent surveillance may be achieved during the operation.

4. High Possibility of Performing a Successful Operation:

Because the angled head (203) of the surgical knife (20) can be moved only along the groove (312), the blade on the head (203) will not cut any proper tissue, especially the motor branch (73) in the palm and so the operation is much probably successful.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A set of surgical instruments for carpal tunnel surgery, comprising:
a group of differently-sized dilators (10, 11, 12, 13) for progressively dilating a carpal tunnel of a patient from the wrist to the palm;
at least one guide (30, 30′) designed to be inserted in said carpal tunnel after proper dilation, said at least one guide (30, 30′) having a trough (31, 31′) defining a first longitudinal groove (311, 311′) and a second longitudinal groove (312, 312′), said trough (31, 31′) being formed with a rear handle (32, 32′) defining a cavity open to said first longitudinal groove (311, 311′);
a group of surgical knives (20, 21) each movable along said second longitudinal groove (312) of said guide (30) for cutting, each of said surgical knives (20, 21) being provided with a shank (201) in a zigzag configuration, said shank (201) having a rear end formed with a grip (202) and a front end formed with an angled head (203).

2. The set of surgical instruments as claimed in claim 1, wherein said second longitudinal groove (312, 312′) extends through the full length of said rear handle (32, 32′).

3. The set of surgical instruments as claimed in claim 1, wherein said group of dilators (10, 11, 12, 13) includes four differently-sized dilators (10, 11, 12, 13), and wherein each of three of said dilators (10, 11, 12, 13) has an oval section.

4. The set of surgical instruments as claimed in claim 3, wherein said group of surgical knives (20, 21) includes a first knife (20) having a straight blade and a second knife (21) having a hooked blade.

5. The set of surgical instruments as claimed in claim 4, wherein said hooked blade of said second knife (21) has a blunt outer edge.
